SEQUENCE LISTING (26 sequences)

SEQ ID NO: 1 - DNA, 4 bp, Artificial Sequence
  AciI recognition sequence
ccgc

SEQ ID NO: 2 - DNA, 6 bp, Artificial Sequence
  AclI recognition sequence
aacgtt

SEQ ID NO: 3 - DNA, 6 bp, Artificial Sequence
  AgeI recognition sequence
accggt

SEQ ID NO: 4 - DNA, 8 bp, Artificial Sequence
  AscI recognition sequence
ggcgcgcc

SEQ ID NO: 5 - DNA, 6 bp, Artificial Sequence
  AvaI recognition sequence
cycgrg

SEQ ID NO: 6 - DNA, 6 bp, Artificial Sequence
  BsaHI recognition sequence
grcgyc

SEQ ID NO: 7 - DNA, 6 bp, Artificial Sequence
  BsiWI recognition sequence
cgtacg

SEQ ID NO: 8 - DNA, 6 bp, Artificial Sequence
  BspDI recognition sequence
atcgat

SEQ ID NO: 9 - DNA, 6 bp, Artificial Sequence
  BsrFI-v2 recognition sequence
rccggy

SEQ ID NO: 10 - DNA, 6 bp, Artificial Sequence
  BssHII recognition sequence
gcgcgc

SEQ ID NO: 11 - DNA, 6 bp, Artificial Sequence
  BstBI recognition sequence
ttcgaa

SEQ ID NO: 12 - DNA, 6 bp, Artificial Sequence
  ClaI recognition sequence
atcgat

SEQ ID NO: 13 - DNA, 6 bp, Artificial Sequence
  EagI recognition sequence
cggccg

SEQ ID NO: 14 - DNA, 4 bp, Artificial Sequence
  HinP1I recognition sequence
gcgc

SEQ ID NO: 15 - DNA, 4 bp, Artificial Sequence
  HpaII recognition sequence
ccgg

SEQ ID NO: 16 - DNA, 4 bp, Artificial Sequence
  HpyCH4IV recognition sequence
acgt

SEQ ID NO: 17 - DNA, 6 bp, Artificial Sequence
  KasI recognition sequence
ggcgcc

SEQ ID NO: 18 - DNA, 6 bp, Artificial Sequence
  MulI recognition sequence
acgcgt

SEQ ID NO: 19 - DNA, 6 bp, Artificial Sequence
  NarI recognition sequence
ggcgcc

SEQ ID NO: 20 - DNA, 6 bp, Artificial Sequence
  NgoMIV recognition sequence
gccggc

SEQ ID NO: 21 - DNA, 8 bp, Artificial Sequence
  NotI recognition sequence
gcggccgc

SEQ ID NO: 22 - DNA, 6 bp, Artificial Sequence
  PaeR7I recognition sequence
ctcgag

SEQ ID NO: 23 - DNA, 7 bp, Artificial Sequence
  RsrII recognition sequence
cggwccg

SEQ ID NO: 24 - DNA, 6 bp, Artificial Sequence
  SalI recognition sequence
gtcgac

SEQ ID NO: 25 - DNA, 8 bp, Artificial Sequence
  SgrAI recognition sequence
crccggyg

SEQ ID NO: 26 - DNA, 6 bp, Artificial Sequence
  TspMI recognition sequence
cccggg

(19) United States
(12) Patent Application Publication
(10) Pub. No.: US 2023/0416832 A1
(43) Pub. Date: Dec. 28, 2023

(54) METHODS FOR DNA METHYLATION ANALYSIS

(71) Applicant: The General Hospital Corporation, Boston, MA (US)

(72) Inventors: A. John Iafrate, Newton, MA (US); Ju Cheng, Malden, MA (US)

(21) Appl. No.: 18/037,899

(22) PCT Filed: Nov. 19, 2021
(86) PCT No.: PCT/US2021/060089

§ 371 Date: May 19, 2023

Related U.S. Application Data
(60) Provisional application No. 63/116,629, filed on Nov. 20, 2020.

(51) Int. Cl. C12Q 1/6886; C12Q 1/6806

(52) U.S. Cl. CPC C12Q 1/6886; C12Q 1/6806; C12Q 2600/154

(57) ABSTRACT

Next Generation Sequencing (NGS)-based methods using Methylation Sensitive Restriction Enzymes (MSREs) for methylation analysis of DNA, e.g., circulating tumor DNA.

### CLAIM OF PRIORITY

This application claims priority the benefit of U.S. Provisional Patent Application Ser. No. 63/116,629, filed on Nov. 20, 2020. The entire contents of the foregoing are hereby incorporated by reference.

### TECHNICAL FIELD

Described herein are Next Generation Sequencing (NGS)-based methods using Methylation Sensitive Restriction Enzymes (MSREs) for methylation analysis of DNA, e.g., circulating tumor DNA.

### BACKGROUND

While great strides have been made in the development of effective targeted and immuno-based cancer therapies, the ultimate key to reducing cancer mortality is to detect tumors early, at a stage when they are small, anatomically limited, and surgically resectable. For certain tumor types large-scale screening programs have been in place including mammography for breast cancer, endoscopy for colorectal and gastric cancers, PSA testing for prostate cancer, and pap smears for cervical cancer. Blood-based biomarker screens such as PSA are easy to obtain and relatively inexpensive to perform, and as a result there is interest in developing additional blood-based screens. Since tumor cells shed DNA into the circulation, detecting DNA somatic mutations in circulating cell-free DNA (cfDNA) with deep sequencing has become clinically used for patient monitoring, and potentially provides a more universal avenue for early stage cancer diagnosis^{1,2}.

Since the majority of cfDNA detected in patients is derived from normal circulating blood cells, it is important to develop sensitive detection methodologies to find the rare tumor DNA fragments. Tumor derived cfDNA can be distinguished from normal cell DNA by its fragment size³, by the presence of DNA mutations^{1,2}, and by the pattern and amount of DNA methylation⁴⁻⁷. Using next generation sequencing (NGS)-based DNA mutation detection, clinical assays have been developed and validated with a limit of detection (LOD), e.g., of about 0.1 to 0.2 or 0.25% tumor cell fraction, that has been shown to be useful in monitoring disease progression and therapy resistance in late-stage cancer patients⁸, however the usefulness of such NGS assays for detecting cancer in early stage patients is currently limited⁹. Other DNA mutation detection methods such as droplet digital PCR have superior LOD but require a priori knowledge of what mutations a tumor may possess, and so are not practical and will fail to detect many tumors.

Analysis of DNA methylation patterns in cfDNA holds great promise as an early-detection method since there are millions of methylated cytosines (me-C) in the genome, these me-Cs are stable in cfDNA, and the patterns of methylated sequences are tissue-specific¹⁰⁻¹². Thus, by quantitating the hundreds of thousands of differentially-methylated sites (DMSs) between cancer-derived DNA and normal cell DNA one can be 1-2 logs more sensitive than mutation detection^{6,13}. By statistically clustering the DMSs from a patient's cfDNA sample with a set of known tumors, one can also determine the tissue of origin of such tumors⁶⁻⁷. Thus, the optimization of a DNA methylation-based cancer detection assay could have potentially great value for the field of cancer diagnostics, and usher in a new paradigm where asymptomatic patients are screened for early and treatable cancers with a powerful blood-based test.

There are multiple methods for analyzing DNA methylation that can be grouped into three categories: bisulfite conversion-based strategies, e.g., whole-genome bisulfite sequencing (WGBS); me-C affinity enrichment-based strategies; and methylation-sensitive restriction enzyme (MSRE)-based strategies¹⁴. Although bisulfite sequencing is the gold standard in detecting CpG methylation in general, the conversion process is highly damaging to DNA and results in significant template loss (and thus loss of sensitivity) and the base diversity is compromised after conversion, resulting in more misalignment or alignment failure. The template loss and alignment issues are especially problematic with the low amounts of short fragments that typifies cfDNA. Given that these assays are not enriched, but require whole-genome sequencing, they are extremely expensive at the depths of sequencing needed for rare cfDNA detection. MSRE-based strategies are the better approach to cfDNA, since they are not damaging to DNA in general, and do not cause non-specific template loss.

### SUMMARY

Described herein are methylation sensitive restriction enzyme (MSRE)-based method for methylation analysis of DNA (e.g., circulating tumor DNA; an exemplary method using circulating tumor DNA is referred to herein as MET-CT). Thus, provided herein are methods comprising (a) providing a sample comprising DNA; (b) generating a first population of blunt-ended fragments from the DNA, (c) digesting the first population of fragments using one or more methylation sensitive restriction enzymes (MSREs), wherein the MSRE leaves a 5′-overhang of at least one nucleotide; (d) filling in the overhangs with modified nucleosides to create a second population of blunt-ended fragments; and (e) purifying fragments comprising modified nucleosides.

In some embodiments, the DNA is cell-free DNA, optionally genomic DNA.

In some embodiments, the first population of fragments are fragments with dA-tails. In some embodiments, generating the first population of fragments from the DNA comprises using mechanical shearing or enzymatic shearing, optionally to obtain fragments of 100 to 1000, e.g., 150-500 or 150-350 nts.

In some embodiments, the modified nucleosides are biotinylated or labeled with digoxigenin. In some embodiments, the modified nucleosides are biotinylated nucleosides and the fragments comprising modified nucleosides are purified using streptavidin. In some embodiments, purifying fragments comprising biotinylated nucleosides using streptavidin comprising contacting the fragments with streptavidin beads.

In some embodiments, the biotinylated nucleosides comprise biotinylated cytidine.

In some embodiments, the MSRE is listed in Table 1. In some embodiments, the MSRE is HpaII, AciI, HinP1I, or HpyCH4IV, preferably wherein the MSRE is HpaII.

In some embodiments, the methods also include after step (d) adding an adenine (A) to the 3′ end of each fragment in the second population of blunt-ended fragments; ligating an adaptor comprising a NGS sequencing primer sequence with a corresponding 5′ thymidine (T) overhang to the ends; and sequencing the purified fragments using next generation sequencing (NGS).

In some embodiments, the methods include using a DNA polymerase to add the adenine to the 3′ end of each fragment. In some embodiments, the DNA polymerase is Klenow exo- or Taq polymerase.

In some embodiments, the sample comprises genomic DNA from a biological sample from a subject.

In some embodiments, the biological sample is a sample comprising tissue, whole blood, plasma, or serum. In some embodiments, the tissue comprises or is suspected to comprise tumor tissue from surgical resection, punch biopsy, needle biopsy, or biopsy.

In some embodiments, the subject has, or is suspected to have, a cancer.

In some embodiments, the methods further include quantifying reads for each sequence.

In some embodiments, the methods further include generating a matrix comprising the quantified reads for each sequence. In some embodiments, the matrix is generated by a method comprising aligning the sequences obtained by a method as described herein with a reference sequence, identifying reads that correspond to known cut sites for the MSRE in the DNA, determining a number of reads for each known cut site, and generating a matrix wherein each data point in the matrix corresponds to the number of reads for each known cut site.

Also provided herein are computer-implemented methods comprising generating, using a computing device, a matrix comprising the quantified reads generated as described herein, wherein each data point in the matrix corresponds to a number of reads for each known cut site for the MSRE in the DNA.

In some embodiments, the matrix is generated by a method comprising aligning the sequences obtained by a method described herein with a reference sequence, identifying reads that correspond to known cut sites for the MSRE in the DNA, determining a number of reads for each known cut site, and generating a matrix wherein each data point in the matrix corresponds to the number of reads for each known cut site.

In some embodiments, the methods further include comparing the matrix to a reference matrix to identify one or more differentially methylated sites (DMSs).

In some embodiments, the sample comprises genomic DNA from a biological sample from a subject, and the reference matrix is a matrix from the same subject at an earlier timepoint, or represents a matrix from a reference subject or cohort of reference subjects.

In some embodiments, the reference subject or cohort of reference subjects are subjects who do not have cancer, who have been diagnosed with cancer, who have responded to a treatment for cancer, who do not have a disease associated with loss of imprinting (LOI); who do have a disease associated with LOI; who do have a condition associated with aberrant methylation, or who do not have a condition associated with aberrant methylation.

Also provided herein are methods for detecting the presence of a condition associated with aberrant methylation in a sample. The methods include generating, preferably using a computing device, a subject matrix comprising quantified reads generated as described herein, wherein the sample comprises genomic DNA from a biological sample from a subject; and (i) comparing, preferably using a computing device, the matrix to a reference matrix that represents a matrix from a subject who does not have a condition associated with aberrant methylation, wherein a significant difference from the reference matrix indicates that the subject has a condition associated with aberrant methylation; or (ii) comparing, preferably using a computing device, the matrix to a reference matrix that represents a matrix from a subject who has condition associated with aberrant methylation, wherein similarity to, or lack of significant difference from, reference matrix indicates that the subject has a condition associated with aberrant methylation; or (iii) comparing, preferably using a computing device, the matrix to a reference matrix from the same subject at an earlier point in time, wherein a difference from the reference matrix indicates that the subject has developed a condition associated with aberrant methylation. In some embodiments, the methods include administering a treatment for the condition associated with aberrant methylation to the subject.

Also provided are methods for detecting the presence of a haploid or diploid methylation in a sample. The methods include aligning sequences obtained by a method as described herein with a reference sequence; categorizing each read as on-target or off-target, wherein on-target reads have at least one-end starting at a cut site, and off-target reads have no ends that start at a cut site; detecting the presence of one or more single nucleotide polymorphisms (SNPs) in the sequences; determining a pattern of SNPs in the on-target and off-target reads; and comparing the pattern of SNPs in the on-target reads to the pattern of SNPs in the off-target reads, wherein the presence of a haploid SNP pattern in the on-target reads and a diploid pattern of off-target reads, indicates that one of the alleles is methylated (silenced, imprinted).

In some embodiments, the methods further include: comparing the pattern of SNPs in the on-target and off-target reads to a reference pattern, and identifying a subject as having a pathological condition associated with aberrant methylation or loss of imprinting when the pattern differs from a reference pattern that represents a normal subject, e.g., SNP pattern of on-target reads is haploid while the pattern of off-target reads is diploid, or matches a reference pattern that represents a subject with a pathological condition associated with aberrant methylation or loss of imprinting, e.g., SNP patterns of on-target reads and off-target reads are both diploid.

Additionally provided herein are methods for detecting methylation in a sample. The methods include generating, preferably using a computing device, a subject matrix comprising quantified reads generated using a method described herein.

In some embodiments, the methods include comparing, preferably using a computing device, the matrix to a reference matrix. In some embodiments, the sample is from a subject, and the reference matrix represents a matrix from a subject who does not have a condition associated with aberrant methylation; represents a matrix from a subject who has condition associated with aberrant methylation; or is a matrix from the same subject at an earlier point in time.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-E. Exemplary embodiments of methods described herein. 1A-B, Fragmented DNA (1A, cfDNA; 1B, sheared gDNA) are end blunted and digested with HpaII. Fragments cut by HpaII are labeled with biotin (dots) during end-repair. All the fragments are ligated to Y-adapters. After streptavidin purification, only fragments with unmethylated HpaII sites are enriched and then amplified for sequencing. 1C-D, Fragmented DNA (1C, cfDNA; 1D, sheared gDNA) are end blunted and end-repaired/dA-tailed before being digested with HpaII. Fragments cut by HpaII are filled in and labeled with biotin (filled ovals). All the fragments are ligated to Y-adapters. After streptavidin purification, only fragments with unmethylated HpaII sites are enriched and then amplified for sequencing. 1E, an exemplary workflow 100.

FIG. 2. In some exemplary embodiments, MET-CT specifically enriches signals at unmethylated HpaII sites. When down-sampled to 30M on-target reads in total, HCT-116 (100% unmethylated) had high coverage (>250×) at MEH1 promoter while RKO (100% methylated) had no coverage.

FIG. 3. Volcano plot of the HpaII sites of lung cancer cell lines versus buffy coat samples. Dark grey dots indicate differentially-methylated sites (DMSs) with the cut-off at p-value oft test <0.01 and fold change >32.

FIG. 4. Predicted sensitivity at different cut-off. X-axis: different FC cut-off. Different lines show different minimal signals need to determine an outlier. Frame area is zoomed in to show the sensitivity below 0.001.

FIG. 5. Classification of tumor and non-tumor samples. Genomic DNA extracted from in vitro cultured tumor cell lines sequenced to generate MET-CT profiles and establish a model to separate tumor and non-tumor samples (PC1) and different tumor types (PC2).

### DETAILED DESCRIPTION

Described herein is a MSRE method for methylation analysis of circulating tumor DNA (an exemplary method is referred to herein as MET-CT). MET-CT is purpose-built for analyzing the methylation status of ctDNA with its limited quantity and very short fragment length. In some embodiments, the methods use next generation sequencing methods. Some embodiments of this assay (one example is MET-CT) are cost effective, by analyzing only genomic sequences cut by HpaII (as opposed to genome-wide bisulfite sequencing, which is >30× as expensive).

This assay can be used, e.g., to analyze real-world plasma samples from multiple early stage cancer patients, e.g., including breast, colon and lung cancers. This methylation-sensitive restriction-enzyme based methylome assay “MET-CT” can be used, e.g., to detect cancer, e.g., early stages of cancer, and accurately classify their tissue of origin. In addition, the methods can be used to detect changes in methylation patterns associated with disease progression and response to epigenetic therapies, and can be used to study mechanisms of treatment resistance, diagnose cancers of unknown primary site, and diagnose other conditions associated with aberrant methylation, e.g., conditions associated with loss of imprinting (LOI) such as Beckwith-Wiedemann Syndrome, Prader-Willi syndrome, and Angelman syndrome; autoimmine diseases such as rheumatoid arthritis (RA), systemic lupus erythematosus (SLE), and multiple sclerosis (MS); metabolic derangements including hyperglycemia (e.g., associated with type I and type II diabetes) and hyperlipidemia (e.g., obesity-related conditions); neurological disorders including autism spectrum disorder (ASD) and Rett Syndrome; and aging. See, e.g., Jin and Liu, Genes Dis. 2018 March; 5(1): 1-8.

This approach can be used with intact genomic DNA and/or small amounts of DNA, e.g., fragmented DNA present in circulation, e.g., cell-free DNA (cfDNA).

The present methods include the use of NGS-based library construction using methylation sensitive restriction enzymes (MSRE).

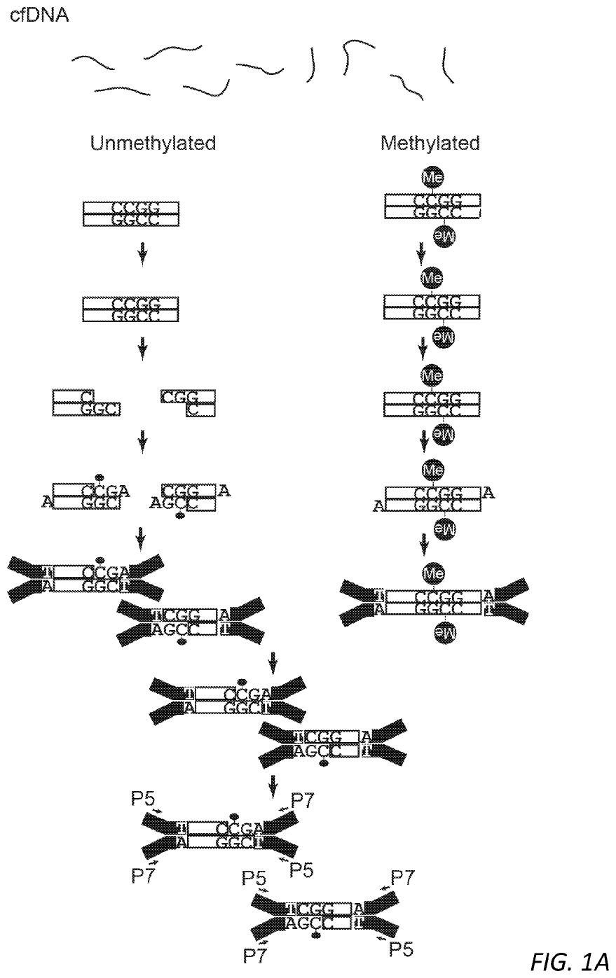

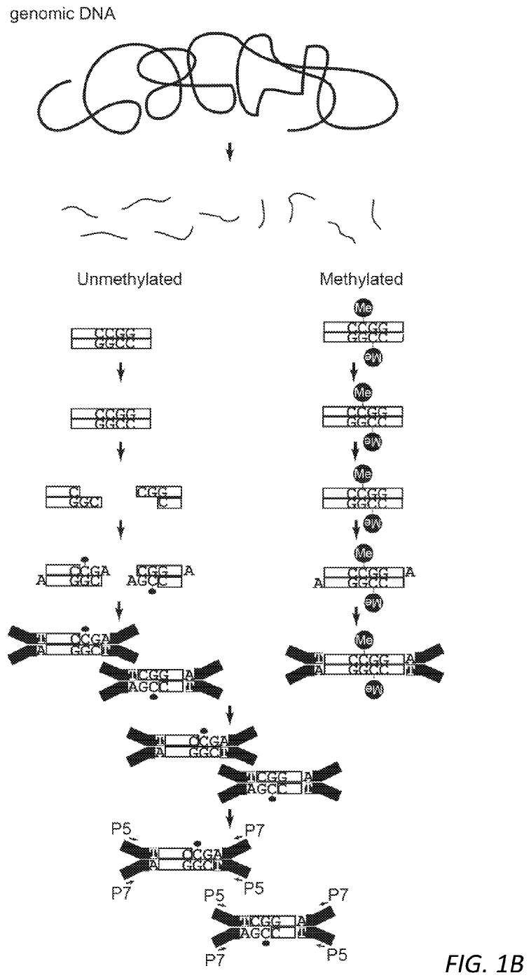

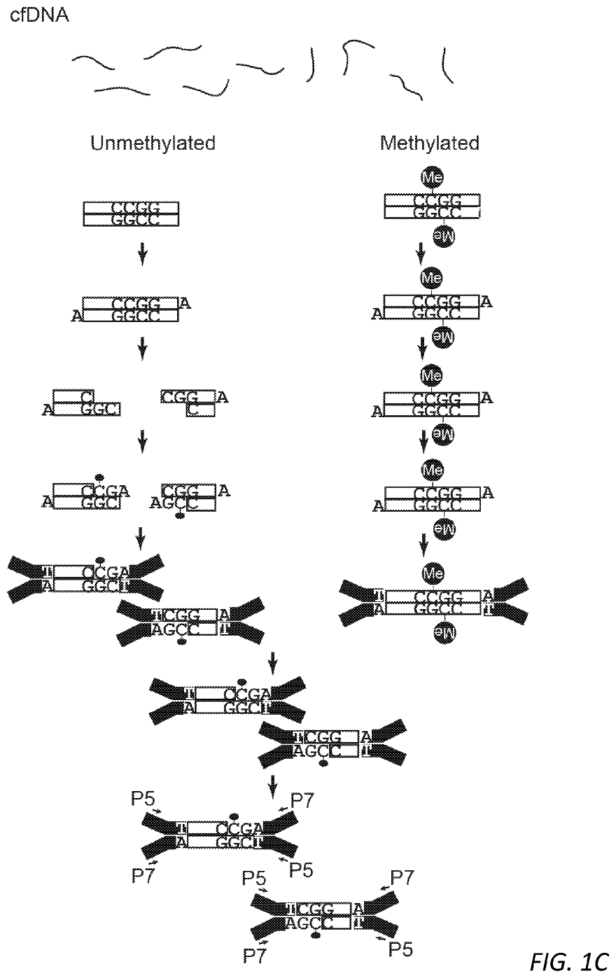

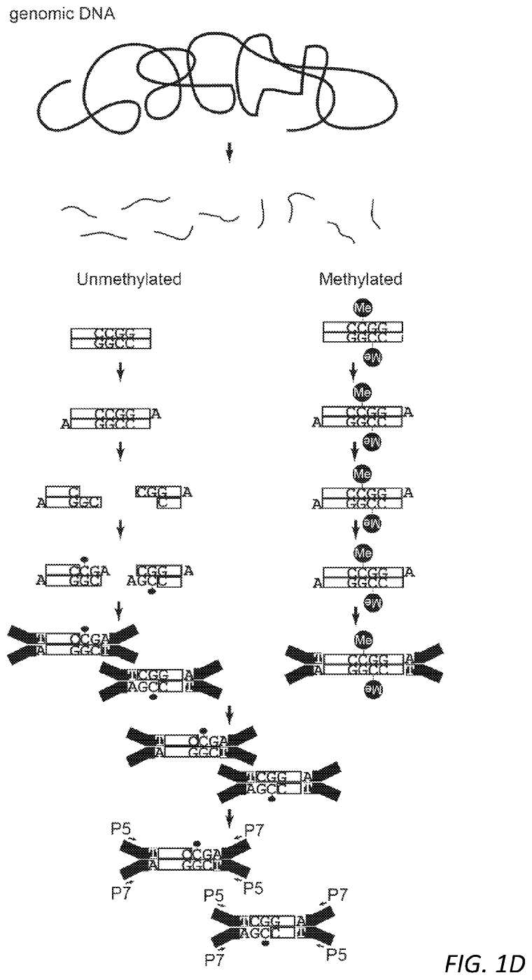

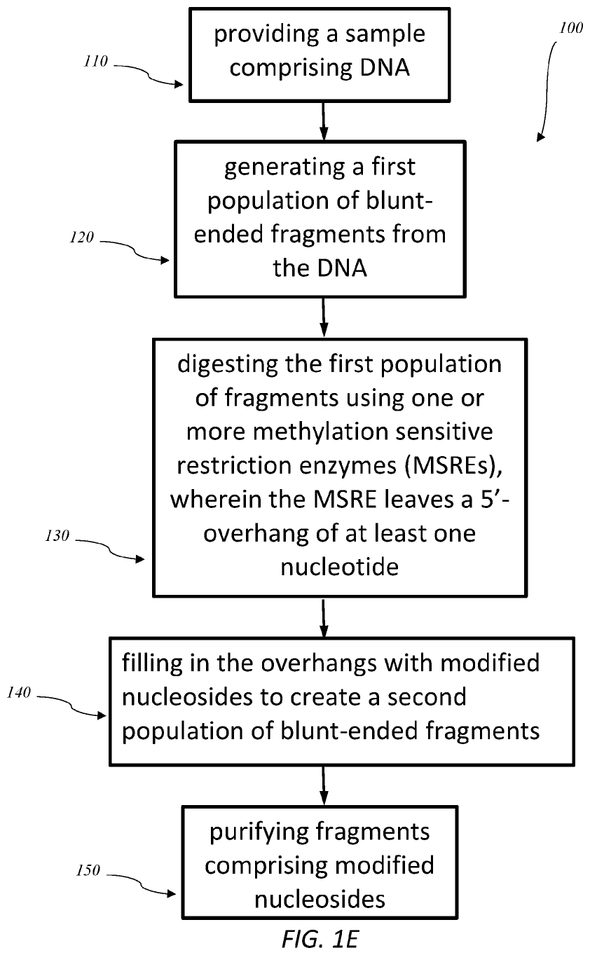

In some embodiments, as shown in FIG. 1A, cfDNA fragments are end-blunted, and then digested by MSRE (HpaII). Fragments with unmethylated MSRE sites (CCGG for HpaII) are cut while methylated MSRE sites remain intact after digestion. Adhesive ends generated by MSRE digestion are filled-in with nucleotides labeled with biotin or digoxigenin. All the fragments are tailed with dATP at both ends, and then ligated to sequencing adapters. Fragments with unmethylated MSRE sites are enriched by biotin/digoxigenin affinity purification. Enriched fragments are ready for sequencing with or without amplification.

In some embodiments, as shown in FIG. 1B, Genomic DNA are fragmented, end-blunted, and then digested by MSRE (HpaII). Fragments with unmethylated MSRE sites (CCGG for HpaII) are cut while methylated MSRE sites remain intact after digestion. Adhesive ends generated by MSRE digestion are filled-in with nucleotides labeled with biotin or digoxigenin. All the fragments are tailed with dATP at both ends, and then ligated to sequencing adapters. Fragments with unmethylated MSRE sites are enriched by biotin/digoxigenin affinity purification. Enriched fragments are ready for sequencing with or without amplification.

In some embodiments, as shown in FIG. 1C, cfDNA fragments are end-blunted, dA-tailed and then digested by MSRE (HpaII). Fragments with unmethylated MSRE sites (CCGG for HpaII) are cut while methylated MSRE sites remain intact after digestion. Adhesive ends generated by MSRE digestion are filled-in with nucleotides labeled with biotin or digoxigenin, and then ligated to sequencing adapters. Fragments with unmethylated MSRE sites are enriched by biotin/digoxigenin affinity purification. Enriched fragments are ready for sequencing with or without amplification.

In some embodiments, as shown in FIG. 1D, genomic DNA are fragmented, end-blunted, dA-tailed and then digested by MSRE (HpaII). Fragments with unmethylated MSRE sites (CCGG for HpaII) are cut while methylated MSRE sites remain intact after digestion. Adhesive ends generated by MSRE digestion are filled-in with nucleotides labeled with biotin or digoxigenin, and then ligated to sequencing adapters. Fragments with unmethylated MSRE sites are enriched by biotin/digoxigenin affinity purification. Enriched fragments are ready for sequencing with or without amplification.

Referring now to the exemplary workflow 100 shown in FIG. 1E, in some embodiments the method includes step 110 of providing a sample comprising DNA; step 120 of generating or obtaining fragments, e.g., using mechanical shearing, and then blunt-ending the fragments or dA-tailing using DNA polymerase. Then in step 130 the fragments are then subjected to digestion with an MSRE. In step 140 the overhangs (GC in the case of HpaII) are then filled in with modified nucleosides, e.g., biotinylated nucleosides (e.g., biotinylated cytidine) or nucleosides labeled with desthiobiotin or digoxigenin, to create blunt-ended fragments; fragments comprising modified nucleosides are then isolated, e.g., purified, in step 150.

In some embodiments, at least one adenine is added to the 3′ ends of each fragment, e.g., using a DNA polymerase such as Taq, and an adaptor comprising a NGS sequencing primer sequence with a 5′ T overhang is ligated to the ends. The reaction products are cleaned up by isolating fragments that include the modified nucleoside, e.g., using avidin, e.g., streptavidin or neutravidin for biotinylated nucleosides, e.g., streptavidin beads, to obtain only those fragments that include biotinylated nucleosides, which can then be identified, e.g., sequenced using NGS. Because only the fragments cut by the MSRE are captured, and only the fragments that are unmethylated are cut by the MSRE, the NGS read coverage correlates with the methylation status, with reads piling up at unmethylated genomic regions. As an alternative to NGS, array or hybridization-based methods can be used, e.g., when the sequence of regions expected to be un-modified is known; these methods can be used, for example, to determine whether specific regions of interest are unmethylated.

The present disclosure exemplifies the use of HpaII, which cuts at C↓CGG sequences but is blocked from cutting the sequence when the cytosines are methylated. CG sequences are important in methylation, as cytosine methylation occurs at CG sequences. There are approximately 2.3 million CCGG sites in the genome, which are enriched in the gene promoters and enhancers where methylation status is functionally critical. By just sequencing the unmethylated CCGG sites, only 2-3% reads are needed as compared to whole-genome bisulfite sequencing (WGBS) without losing information. By avoiding bisulfite conversion, template loss is minimized, and base diversity is kept, resulting in higher quality and quantity libraries. There have been library construction methods described using DNA adapters ligated specifically to the CG cut-site cohesive ends.^{15,16} Compared to those methods, MET-CT has the advantage of avoiding aberrant ligation events between random cfDNA molecules and self-ligation of the adapters, which in the end results in more on-target sequencing reads in the library, and ultimately a much lower sequencing cost.

As an alternative or in addition to HpaII, other MSREs can also be used. A number of such enzymes are known in the art, including those listed in Table 1; engineered MSREs can also be used. Four-base cutters (e.g., those in bold in Table 1) are preferred for the present methods since there are more cut sites, so more detectible events per genome. Thus in some embodiments, the MSRE is HpaII, AciI, HinP1I, or HpyCH4IV. However, the other MSREs, or combinations of one or more MSREs, can also be used.

TABLE 1
Methylation-Sensitive Restriction Enzymes

| Restriction | Enzyme recognition | |
|---|---|---|
| enzyme | sequence | SEQ ID NO: |
| AciI | C↓CGC | 1. |
| AclI | AA↓CGTT | 2. |
| AgeI | A↓CCGGT | 3. |
| AscI | GG↓CGCGCC | 4. |
| AvaI | C↓YCGRG | 5. |
| BsaHI | GR↓CGYC | 6. |
| BsiWI | C↓GTACG | 7. |
| BspDI | AT↓CGAT | 8. |
| BsrFI-v2 | R↓CCGGY | 9. |
| BssHII | G↓CGCGC | 10. |
| BstBI | TT↓CGAA | 11 |
| ClaI | AT↓CGAT | 12. |
| EagI | C↓GGCCG | 13. |
| HinP1I | G↓CGC | 14. |
| HpaII | C↓CGG | 15. |
| HpyCH4IV | A↓CGT | 16. |
| KasI | G↓GCGCC | 17. |
| MulI | A↓CGCGT | 18 |
| NarI | GG↓CGCC | 19. |
| NgoMIV | G↓CCGGC | 20. |
| NotI | GC↓GGCCGC | 21. |
| PaeR7I | C↓TCGAG | 22. |
| RsrII | CG↓GWCCG | 23. |
| SalI | G↓TCGAC | 24 |
| SgrAI | CR↓CCGGYG | 25. |
| TspMI | C↓CCGGG | 26. |

The methods can be performed on any bodily tissue or fluid sample. As used herein the term “sample”, when referring to the material to be tested for the presence of a biological marker using the method of the invention, includes inter alia tissue (e.g., tumor tissue from surgical resection, punch biopsy, needle biopsy, or biopsy), whole blood, plasma, serum, urine, sweat, saliva, exosome or exosome-like microvesicles (U.S. Pat. No. 8,901,284), lymph, feces, cerebrospinal fluid, ascites, bronchoalveolar lavage fluid, pleural effusion, seminal fluid, sputum, nipple aspirate, post-operative seroma, or wound drainage fluid. The type of sample used may vary depending upon the clinical situation in which the method is used.

Various methods are well known within the art for the identification and/or isolation and/or purification of a biological marker from a sample. An “isolated” or “purified” biological marker is substantially free of cellular material or other contaminants from the cell or tissue source from which the biological marker is derived i.e. partially or completely altered or removed from the natural state through human intervention. For example, nucleic acids contained in the sample are first isolated according to standard methods, for example using lytic enzymes, chemical solutions, or isolated by nucleic acid-binding resins following the manufacturer's instructions.

Cancer

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genitourinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CIVIL) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

Matrices

Once the sequences have been obtained, a matrix can be generated that represents the level of methylation (based on the number of NGS reads) present at each methylation sequence site in the sample, quantitating and providing a profile of methylation in the sample. These matrices can be analyzed and compared with reference matrices to identify DMSs. The matrices can be generated by exporting the (normalized) counts of the reads starting at each of the cut sites across the reference genome, e.g., the (normalized) counts at HpaII sites across human genome, such that each data point in the matrix corresponds to a specific known cut site. In some embodiments, reads generated by next generation sequencing are aligned to the reference genome (e.g., hg19) with an aligner (e.g., Bowtie, BWA MEM, NovoAlign). Counts for each of the known cut sites are used to generate the matrix.

Standard computing devices and systems can be used and implemented to generate the matrices described herein. Computing devices include various forms of digital computers, such as laptops, desktops, mobile devices, workstations, personal digital assistants, servers, blade servers, mainframes, and other appropriate computers. In some embodiments, the computing device is a mobile device, such as personal digital assistant, cellular telephone, smartphone, tablet, or other similar computing device. The components described herein, their connections and relationships, and their functions, are meant to be exemplary only, and are not meant to limit implementations of the inventions described and/or claimed in this document.

Computing devices typically include one or more of a processor, memory, a storage device, a high-speed interface connecting to memory and high-speed expansion ports, and a low speed interface connecting to low speed bus and storage device. Each of the components are interconnected using various busses, and can be mounted on a common motherboard or in other manners as appropriate. The processor can process instructions for execution within the computing device, including instructions stored in the memory or on the storage device to display graphical information for a GUI on an external input/output device, such as a display coupled to a high speed interface. In other implementations, multiple processors and/or multiple buses can be used, as appropriate, along with multiple memories and types of memory. Also, multiple computing devices can be connected, with each device providing portions of the operations (e.g., as a server bank, a group of blade servers, or a multi-processor system).

The computing device can generate the matrix and provide it to an end user, e.g., a health care provider, by display on a screen or via providing a printed output.

In addition, the methods can include comparing matrices between a subject (e.g., a subject or test matrix) and a reference matrix to identify DMSs. A low number of DMSs (e.g., a number below a threshold number of DMSs) can indicate similarity to (e.g., lack of significant difference from) a reference matrix, while a number of DMSs above a threshold number can indicate difference (e.g., significant difference) from the reference. The reference matrix can be, e.g., a reference matrix generated from (and representing) a cohort of control or disease subjects (e.g., from subjects with tumors), or from the same subject at an earlier or later time point; the matrices can represent a baseline, pre-treatment, during treatment, or post-treatment profile of methylation in the sample. Similarity to a disease reference matrix or difference from a healthy control reference matrix can indicate the presence of or high risk of developing a disease, while difference from the disease matrix and similarity to a healthy control can indicate likely absence of or low risk of developing the disease, where high or low risk is as compared to the risk level in a reference cohort, e.g., the general population. In some embodiments, the methods can be used to detect alterations in methylation patterns in a subject who is being treated with a treatment that alters methylation, e.g., chemotherapy (e.g., with platinum-based, methyl transferase inhibitors and other chemotherapeutics); comparisons can be made to a matrix that represents successful treatment, e.g., tumor shrinkage or suppression, or unsuccessful treatment, e.g., tumor growth or metastasis. The comparisons can be made using methods known in the art.

Suitable reference DMSs, or levels of DMSs, can be determined using methods known in the art, e.g., using standard clinical trial methodology and statistical analysis. The reference values can have any relevant form. In some cases, the reference comprises a predetermined DMS or value for a meaningful level of DMSs, e.g., a control reference level that represents a normal DMS or level of DMSs, e.g., a level that represents normal human variation and thus is similar to or not different from methylation in an unaffected subject or a subject who is not at risk of developing a disease described herein, and/or a disease reference that represents a DMS level of DMSs associated with conditions of aberrant methylation as described herein.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a control reference subject does not have a disorder described herein.

A disease reference subject is one who has (or has an increased risk of developing) a disorder described herein. An increased risk is defined as a risk above the risk of subjects in the general population.

Thus, in some cases the level of DMSs in a subject being more than or equal to a reference level of DMSs is indicative of a clinical status (e.g., indicative of a disorder as described herein). In other cases the level of DMSs in a subject being less than or equal to the reference level of DMSs is indicative of the absence of disease or normal risk of the disease. In some embodiments, the amount by which the level in the subject is the less than the reference level is sufficient to distinguish a subject from a control subject, and optionally is a statistically significantly less than the level in a control subject. In cases where the level of DMSs in a subject being equal to the reference level of DMSs, the “being equal” refers to being approximately equal (e.g., not statistically different). In some embodiments, instead of or in addition to a level of DMSs, a score that is calculated based on the methylation status across the DMS may be used.

The predetermined value can depend upon the particular population of subjects (e.g., human subjects) selected. For example, an apparently healthy population will have a different ‘normal’ range of levels of DMSs than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

As one example, bioinformatics analysis of MET-CT can be used to build a tumor detector algorithm, tumor type classifier, and a tumor fraction calculator.

The tumor detector algorithm allows determination of whether or not cancer-derived DNA is present in a specimen and can be developed, for example, using the union of all DMSs across tumor types (e.g., as describe in Example 2, below) to maximize detection rate. The read count statistics can be defined that indicate the presence of tumor. The DMS read counts in the tumor samples are summed and compared to the count in normal to calculate a z-score:

$$z = \frac{x - μ\_c}{σ\_c} ,$$

where x is the sum of the read counts of an unknown sample across all the DMS in a simple linear model, μ_c is the mean value of read counts of normal controls, σ_c is the standard deviation of the read counts distribution for normal controls. The larger the z-score is, the more confidence there is to call the presence of a tumor. In some embodiments, a conservative cut-off z-score >3 can be used as a cutoff to make a positive assay call. The cutoff z-score can be validated in a clinical cohort.

In parallel, DMSs subsets unique to individual tumor types can be used to build a tumor type classifier. Once a sample is identified as positive for cancer, the classifier can be used to determine the probability that that sample is lung, breast or colorectal cancer. For example, the probability of a blood sample containing lung cancer DNA can be predicted with a function t_lung=f(t1), where t_lung is the tumor type “lung” score and t1 is the lung-specific DMS set. Tumor type scores are separately calculated for breast and colon (t_breast and t_colon). In some embodiments, if the probability of one of the tumor types is >95% a specific diagnosis can be made. The cutoff can be validated in the clinic and an ROC curve generated to optimize diagnostic yield. Additional supervised statistical tools/machine learning methods (e.g., multiple linear regression, random forest, support vector machine) can alternatively be applied.

A tumor fraction can be calculated for positive samples that utilizes the larger subset of DMSs for a given tumor (e.g. t1, t2, t4, t5=lung cancer) to maximize the number of sizes for calculation and improve the accuracy. The tumor fraction θ will be calculated as

$$θ = \frac{x - μ\_c}{μ\_t - μ\_c}$$

where x is the minimal read counts needed to call a tumor positive sample, μ_t is the mean of read counts sum at tumor type relevant DMS in tumor cell lines and μ_c is the mean of read counts sum at tumor type relevant DMS in normal controls.

The methods can also be used to identify the loss of imprinting (LOI) in a sample, e.g., for diagnosis of a disease associated with LOI. In some embodiments, LOI is detected by analysis of methylation patterns and single nucleotide polymorphisms (SNPs). SNPs can be identified, e.g., using the NGS reads by the invented method itself, using the off-target reads, or generated by another method (e.g., microarray, WGS). For example, in some embodiments, once reads are generated by the sequencer, they are aligned to the reference genome. Reads are grouped into two categories: on-target and off-target. The on-target reads have at least one-end starting at a cut site. Neither end of the off-target reads starts at a cut site. The on-target reads are unmethylated, while the off-target reads can be either methylated or unmethylated. SNPs are called using on- and off-target reads respectively. If the SNP pattern of on-target reads is haploid while the pattern of off-target reads is diploid, it means that one of the alleles is methylated (silenced, imprinted). For some genomic sites, one of the alleles is silenced (imprinted) by methylation in normal subjects. In some pathological conditions, both alleles are unmethylated (loss of imprinting) at those sites. This can be detected by determining that the SNP pattern of on-target reads is diploid.

Kits

Also provided herein are kits for use in performing a method described herein. The kits can include some or all of: an MSRR, end repair reagents (e.g., T4 DNA polymerase, or Klenow exo-polymerase, or a mixture thereof), biotinylated deoxynucleotide triphosphate and other non-labeled deoxynucleotide triphosphate for fill-in, A-tailing reagents (e.g., adenine and Taq polymerase), adaptors, PCR reagents, streptavidin or other beads to pull down biotin-containing fragments or beads coated by anti-biotin antibodies, and optionally analysis software for generating methylation matrix profiles as described herein.

### EXAMPLES

The invention is further described in the following examples, which do not limit the scope of the invention described in the claims.

### Example 1. Methylation Analysis of Circulating Tumor DNA (MET-CT)

As shown in FIGS. 1A-E, MET-CT uses MSRE methodology utilizing the HpaII restriction enzyme, and allows genome-wide mapping of DNA methylation patterns. HpaII recognizes the sequence CCGG but is blocked from cutting the sequence when the cytosines are methylated. The method takes cfDNA digested with HpaII, then the CG 5′ overhangs are filled in with biotinylated dCTP and free dGTP. The next step uses streptavidin to pull down only the digested (and thus unmethylated) HpaII containing sequences in the genome.

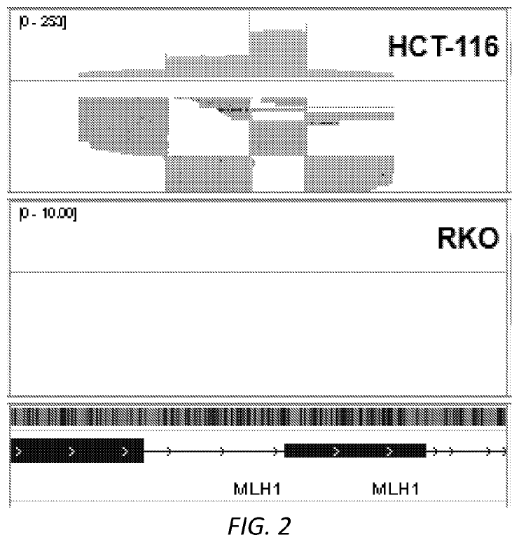

FIG. 2 shows pilot data from two colon cancer cell lines with known methylation status at the MLH1 promoter (HCT116 unmethylated; RKO fully methylated). As expected, MET-CT reads were enriched at the MEH1 promoter in HCT-116 (with a read coverage >250×) but not in RKO (zero reads). Overall assay performance was tested with 24M reads from these two lines, plus analysis of two lung cancer lines (H1975 and HCC827), and two lung cancer cfDNA samples. Since it is critical to subtract methylation profiles contributed by blood cells, which in healthy people comprise the majority of the cfDNA fragments, we generated MET-CT read counts at the 2.3M HpaII sites with six normal buffy coat DNA samples.

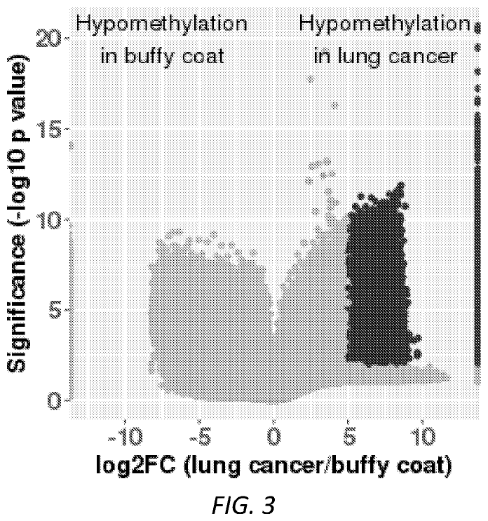

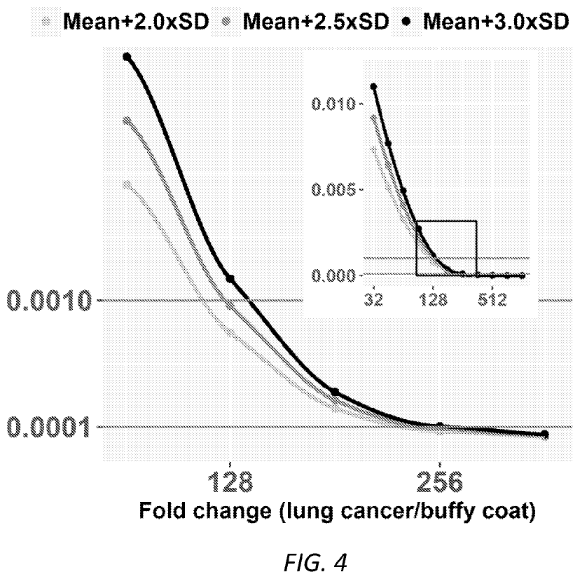

This dataset was used to define the DMSs between lung tumor samples and normal controls, by calculating the read count fold change (FC) between tumor and normal at each HpaII site and performing a t test to define sites with significant differences. HpaII site data is plotted in FIG. 3, with red highlighted dots showing those sites with a >32 fold read count enrichment in tumor vs. normal and a p-value of <0.01, which result in defining over 100,000 DMSs for lung cancer. The assay sensitivity can be predicted by modeling for a tumor how many reads at these sites would be significantly enriched vs. the observed assay background reads in the normal samples. Above-assay background was modeled for 2, 2.5, and 3 SD for these DMSs in FIG. 4. When the fold-change cut-off is set to be >256, the sensitivity was improved to be able to detect less than a 0.0001 tumor fraction, which is in the range needed for an early detection assay.

### Example 2. Generate MET-CT Profiles with Cancer Cell Lines and Defining DMSs

This example focuses initially on the three most common causes of cancer mortality in the United States: lung, colon and breast cancer. MET-CT profiles are built for different tumor types by sequencing a number of cell lines from each tumor type, including histologically-defined but genetically diverse lines. To discriminate MET-CT signals contributed from normal blood cells, which contribute most of the cfDNA in healthy people, additional buffy coat DNA samples are analyzed as normal controls. As per the current MET-CT protocol, DNA extracted from each line/sample will be sheared, end-repaired, digested with HpaII, and labeled with biotin-dCTP. Following streptavidin pull-down, Illumina sequencing adapters including molecular barcodes (UMIs) are ligated on. Libraries will be sequenced. Unique UM-defined sequencing reads initiating at HpaII sites are quantified across all 2.3M HpaII sites to build sample-specific MET-CT profiles.

The MET-CT profiles are compared between the tumor cell lines and the blood samples to establish DMSs for the development of analysis tools including a tumor detector, a tumor type classifier, and a tumor fraction calculator. With this larger set of cell line data, statistical analysis (e.g., t test or ANOVA followed by post-hoc tests) is performed to generate volcano plots of the HpaII sites for each tumor type and define the DMSs by adjusting p-value cut-offs and fold-change cut-off to achieve largest window between tumor samples and normal controls.

Each tumor type will have its own set of DMSs that distinguish it from normal. These tumor sets may overlap, so one may define DMS subsets that are tumor specific (i.e. t1, t3, t7), that only would support one tumor type (e.g. t1, t2, t4, t5=lung), or that define all cancers vs. normal (i.e. union of t1-t7).

For example, classification of tumor and non-tumor samples was performed as follows. Genomic DNA was extracted from in vitro cultured tumor cell lines (breast cancer (n=3), lung cancer (n=3), and colorectal cancer (n=2)), and buffy coat (n=7) were sequenced to generate MET-CT profile. An unsupervised PCA was performed using the MET-CT profiles across the buffy coat and tumor cell lines to establish a model (Classification model/PCA model) to separate tumor and non-tumor samples (PC1) and different tumor types (PC2). MET-CT profiles generated from cfDNA extracted from blood donated by non-tumor individuals (n=7) and breast cancer patients (n=9) were fit into the PCA model.

In order to define tumor type wedges, we calculated the slope of the samples to the geometric mean of the buffycoat samples (non-tumor). The slopes of the three breast cancer cell lines are 0.08, 0.21, 0.60, those of the three lung cancer cell lines are −0.30, 0.43, −0.45, those of the two colorectal cancer cell lines are −0.52 and −0.75. The calculated slopes for the cfDNA samples from breast cancer patients are 0.47, 0.50, 0.53, 0.43, 0.74, 0.64, 0.61, 0.70, and 0.52. They are all above zero and in the same range as the breast cancer tumor cell lines.

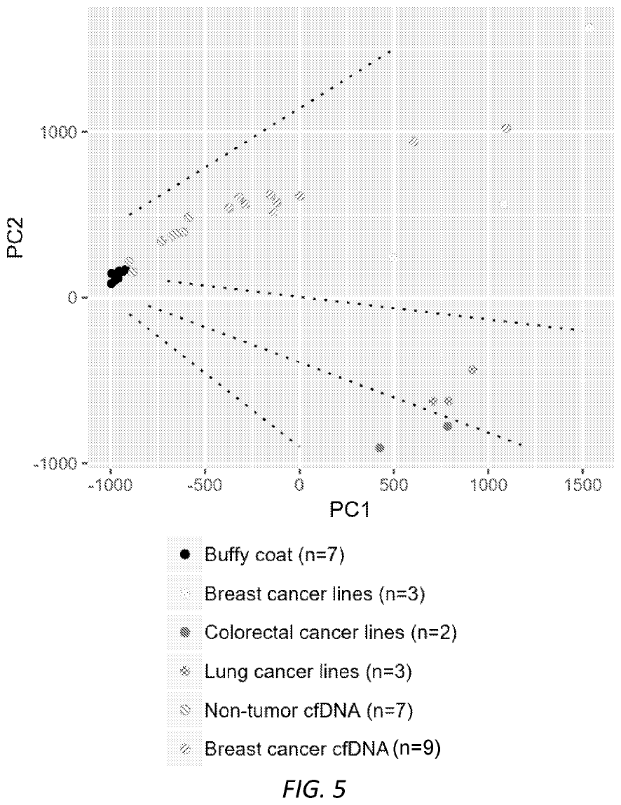

The results, shown in FIG. 5, show that cfDNA from non-tumor individuals were clustered together with the buffy coat while cfDNA from breast cancer patients fell into the breast cancer wedge.

### Example 3. Clinical Validation of MET-CT for Early-Stage Cancer Screening

4A. Generate MET-CT Profiles with Blood Samples Obtained at the Time of Diagnosis (Untreated Patients) with Early-Stage Cancer, as Well as from Healthy Donors.

Clinical validation focuses on analyzing patient blood samples drawn at the time of diagnosis (untreated patients) with early-stage cancer. Analysis of two mutation-positive cfDNA samples with 20 ng input has been completed, yielding ˜200× unique coverage with 40M reads. Thus, the MET-CT wet-lab procedure is compatible with real-world liquid biopsies. Blood samples are collected from patients with tumors e.g., lung, colon, and breast tumors. MET-CT is performed on plasma samples from patients for each of the three tumor types, as well as samples from healthy donors. 10 cc blood samples are collected in EDTA blood tubes, and processed within 3 hours, with nucleic acid extraction from the plasma fraction using the Maxwell ccfDNA extraction kit (Promega). 10-20 ng of cfDNA is used per sample for MET-CT to achieve the LOD at 1/20,000 detection limit with >100,000 DMSs.

4B. Analyze MET-CT Profiles of Early-Stage Tumor Patients and Healthy Donors to Assess MET-CT Clinical Performance.

Sequencing reads generated in 4A are analyzed with the tumor detector, tumor type classifier and tumor fraction calculators sequentially using the DMS defined as described above to allow assessment of MET-CT performance with clinical samples. Reproducibility is assessed by testing in duplicate or triplicate samples that have sufficient cfDNA yields, or for whom multiple blood tubes can be safely obtained. Due to cancer clonal heterogeneity, the MET-CT profiles of real plasma cfDNA might be significantly different from those determined by cancer cell lines. Thus the samples are grouped into training sets and test sets and the training set is used to redetermine DMS and cutoffs by performing negative binomial regression analysis, or to implement previous experience¹⁷ to perform supervised machine learning to improve the accuracy of MET-CT analysis tools. Assay performance is redetermined accordingly.

### REFERENCES

1. Phallen J, Sausen M, Adleff V, et al: Direct detection of early-stage cancers using circulating tumor DNA. Sci Transl Med 9, 2017
2. Cohen J D, Li L, Wang Y, et al: Detection and localization of surgically resectable cancers with a multi-analyte blood test. Science 359:926-930, 2018
3. Mouliere F, Chandrananda D, Piskorz A M, et al: Enhanced detection of circulating tumor DNA by fragment size analysis. Sci Transl Med 10, 2018
4. Chan K C, Jiang P, Chan C W, et al: Noninvasive detection of cancer-associated genome-wide hypomethylation and copy number aberrations by plasma DNA bisulfite sequencing. Proc Natl Acad Sci USA 110:18761-8, 2013
5. Guo S, Diep D, Plongthongkum N, et al: Identification of methylation haplotype blocks aids in deconvolution of heterogeneous tissue samples and tumor tissue-of-origin mapping from plasma DNA. Nat Genet 49:635-642, 2017
6. Shen S Y, Singhania R, Fehringer G, et al: Sensitive tumour detection and classification using plasma cell-free DNA methylomes. Nature 563:579-583, 2018
7. Kang S, Li Q, Chen Q, et al: CancerLocator: non-invasive cancer diagnosis and tissue-of-origin prediction using methylation profiles of cell-free DNA. Genome Biol 18:53, 2017
8. Cheng J, Cao Y, MacLeay A, et al: Clinical validation of a cell-free DNA gene panel. J Mol Diagn, 2019
9. Bettegowda C, Sausen M, Leary R J, et al: Detection of circulating tumor DNA in early- and late-stage human malignancies. Sci Transl Med 6:224ra24, 2014
10. Laird P W: The power and the promise of DNA methylation markers. Nat Rev Cancer 3:253-66, 2003
11. Jones P A: Functions of DNA methylation: islands, start sites, gene bodies and beyond. Nat Rev Genet 13:484-92, 2012
12. Moss J, Magenheim J, Neiman D, et al: Comprehensive human cell-type methylation atlas reveals origins of circulating cell-free DNA in health and disease. Nat Commun 9:5068, 2018
13. Li W, Li Q, Kang S, et al: CancerDetector: ultrasensitive and non-invasive cancer detection at the resolution of individual reads using cell-free DNA methylation sequencing data. Nucleic Acids Res 46: e89, 2018
14. Olkhov-Mitsel E, Bapat B: Strategies for discovery and validation of methylated and hydroxymethylated DNA biomarkers. Cancer Med 1:237-60, 2012
15. Oda M, Glass J L, Thompson R F, et al: High-resolution genome-wide cytosine methylation profiling with simultaneous copy number analysis and optimization for limited cell numbers. Nucleic Acids Res 37:3829-39, 2009
16. Brunner A L, Johnson D S, Kim S W, et al: Distinct DNA methylation patterns characterize differentiated human embryonic stem cells and developing human fetal liver. Genome Res 19:1044-56, 2009
17. Zomnir M G, Lipkin L, Pacula M, et al: Artificial Intelligence Approach for Variant Reporting. JCO Clin Cancer Inform 2, 2018

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method comprising:
(a) providing a sample comprising DNA;
(b) generating a first population of blunt-ended fragments from the DNA,
(c) digesting the first population of fragments using one or more methylation sensitive restriction enzymes (MSREs), wherein the MSRE leaves a 5′-overhang of at least one nucleotide;
(d) filling in the overhangs with modified nucleosides to create a second population of blunt-ended fragments; and
(e) purifying fragments comprising modified nucleosides.

2. The method of claim 1, wherein the DNA is cell-free DNA or genomic DNA.

3. The method of claim 1, wherein the first population of fragments are blunt-ended fragments with dA-tails.

4. The method of claim 1, wherein generating the first population of fragments from the DNA comprises using mechanical shearing or enzymatic shearing, optionally to obtain fragments of 100 to 1000, preferably 150-500 or 150-350 nts.

5. The method of claim 1, wherein the modified nucleosides are biotinylated or labeled with digoxigenin.

6. The method of claim 5, wherein the modified nucleosides are biotinylated nucleosides and the fragments comprising modified nucleosides are purified using streptavidin.

7. The method of claim 6, wherein purifying fragments comprising biotinylated nucleosides using streptavidin comprising contacting the fragments with streptavidin beads.

8. The method of claim 6, wherein the biotinylated nucleosides comprise biotinylated cytidine.

9. The method of claim 1, wherein the MSRE is listed in Table 1.

10. The method of claim 9, wherein the MSRE is HpaII, AciI, HinP1I, or HpyCH4IV, preferably wherein the MSRE is HpaII.

11. The method of claim 1, further comprising after step (d) adding an adenine (A) to the 3′ end of each fragment in the second population of blunt-ended fragments;
ligating an adaptor comprising a NGS sequencing primer sequence with a corresponding 5′ thymidine (T) overhang to the ends; and
sequencing the purified fragments using next generation sequencing (NGS).

12. The method of claim 11, comprising using a DNA polymerase to add the adenine to the 3′ end of each fragment.

13. The method of claim 12, wherein the DNA polymerase is Klenow exo- or Taq polymerase.

14. The method of claim 1, wherein the sample comprises genomic DNA from a biological sample from a subject.

15. The method of claim 14, wherein the biological sample is a sample comprising tissue, whole blood, plasma, or serum.

16. The method of claim 15, wherein the tissue comprises or is suspected to comprise tumor tissue from surgical resection, punch biopsy, needle biopsy, or biopsy.

17. The method of claim 14, wherein the subject has, or is suspected to have, a cancer.

18. The method of claim 11, further comprising quantifying reads for each sequence.

19. The method of claim 18, further comprising generating a matrix comprising the quantified reads for each sequence.

20. The method of claim 19, wherein the matrix is generated by a method comprising aligning the sequences obtained by the method of claim 11 with a reference sequence, identifying reads that correspond to known cut sites for the MSRE in the DNA, determining a number of reads for each known cut site, and generating a matrix wherein each data point in the matrix corresponds to the number of reads for each known cut site.

21. A computer-implemented method, comprising generating, using a computing device, a matrix comprising the quantified reads obtained by the method of claim 18, wherein each data point in the matrix corresponds to a number of reads for each known cut site for the MSRE in the DNA.

22. The method of claim 21, wherein the matrix is generated by a method comprising aligning the sequences obtained by the method of claim 11 with a reference sequence, identifying reads that correspond to known cut sites for the MSRE in the DNA, determining a number of reads for each known cut site, and generating a matrix wherein each data point in the matrix corresponds to the number of reads for each known cut site.

23. The method of claim 21, further comprising comparing the matrix to a reference matrix to identify one or more differentially methylated sites (DMSs).

24. The method of claim 23, wherein the sample comprises genomic DNA from a biological sample from a subject, and the reference matrix is a matrix from the same subject at an earlier timepoint, or represents a matrix from a reference subject or cohort of reference subjects.

25. The method of claim 24, wherein the reference subject or cohort of reference subjects are subjects who do not have cancer, who have been diagnosed with cancer, who have responded to a treatment for cancer, who do not have a disease associated with loss of imprinting (LOI); who do have a disease associated with LOI; who do have a condition associated with aberrant methylation, or who do not have a condition associated with aberrant methylation.

26. A method for detecting the presence of a condition associated with aberrant methylation in a sample, the method comprising:
generating, preferably using a computing device, a subject matrix comprising the quantified reads obtained by the method of claim 18, wherein the sample comprises genomic DNA from a biological sample from a subject; and
(i) comparing, preferably using a computing device, the matrix to a reference matrix that represents a matrix from a subject who does not have a condition associated with aberrant methylation, wherein a significant difference from the reference matrix indicates that the subject has a condition associated with aberrant methylation; or
(ii) comparing, preferably using a computing device, the matrix to a reference matrix that represents a matrix from a subject who has condition associated with aberrant methylation, wherein similarity to, or lack of significant difference from, reference matrix indicates that the subject has a condition associated with aberrant methylation; or
(iii) comparing, preferably using a computing device, the matrix to a reference matrix from the same subject at an earlier point in time, wherein a difference from the reference matrix indicates that the subject has developed a condition associated with aberrant methylation.

27. A method for detecting the presence of a haploid or diploid methylation in a sample, the method comprising:
aligning sequences obtained by the method of claim 11 with a reference sequence;
categorizing each read as on-target or off-target, wherein on-target reads have at least one-end starting at a cut site, and off-target reads have no ends that start at a cut site;
detecting the presence of one or more single nucleotide polymorphisms (SNPs) in the sequences;
determining a pattern of SNPs in the on-target and off-target reads; and
comparing the pattern of SNPs in the on-target reads to the pattern of SNPs in the off-target reads, wherein the presence of a haploid SNP pattern in the on-target reads and a diploid pattern of off-target reads, indicates that one of the alleles is methylated (silenced, imprinted).

28. The method of claim 27, further comprising comparing the pattern of SNPs in the on-target and off-target reads to a reference pattern, and identifying a subject as having a pathological condition associated with aberrant methylation or loss of imprinting when the pattern differs from a reference pattern that represents a normal subject, e.g., SNP pattern of on-target reads is haploid while the pattern of off-target reads is diploid, or matches a reference pattern that represents a subject with a pathological condition associated with aberrant methylation or loss of imprinting, e.g., SNP patterns of on-target reads and off-target reads are both diploid.

29. The method of claim 26 or 28, further comprising administering a treatment for the condition associated with aberrant methylation to the subject.

30. A method for detecting methylation in a sample, the method comprising:
generating, preferably using a computing device, a subject matrix comprising the quantified reads obtained by the method of claim 18.

31. The method of claim 30, further comprising comparing, preferably using a computing device, the matrix to a reference matrix.

32. The method of claim 31, wherein the sample is from a subject, and the reference matrix represents a matrix from a subject who does not have a condition associated with aberrant methylation; represents a matrix from a subject who has condition associated with aberrant methylation; or is a matrix from the same subject at an earlier point in time.
